(19) United States
(12) Patent Application Publication
(10) Pub. No.: US 2004/0028694 A1
(43) Pub. Date: Feb. 12, 2004

(54) Novel nanoparticles and use thereof

(76) Inventors: Mark J. Young, Bozeman, MT (US); Trevor Douglas, Bozeman, MT (US); Yves Idzerda, Bozeman, MT (US)

Correspondence Address: Robin M. Silva, Dorsey & Whitney LLP, Intellectual Property Department, Four Embarcadero Center, Suite 300, San Francisco, CA, 94111-4187, US

(21) Appl. No.: 10/358,089

(22) Filed: Feb. 3, 2003

Related U.S. Application Data
(60) Provisional application No. 60/352,841, filed on Feb. 1, 2002.
(60) Provisional application No. 60/352,842, filed on Feb. 1, 2002.
(60) Provisional application No. 60/352,843, filed on Feb. 1, 2002.

(51) Int. Cl. A61K 39/02; A61K 9/14; C07K 14/195

(52) U.S. Cl. 424/190.1; 424/489; 530/400

(57) ABSTRACT

The present invention is directed to novel compositions and methods utilizing nanoparticles comprising protein cages and cores.

This application claims continuing status and priority under §119/120 of 35 U.S.C. to U.S. S. Nos. 60/352,843, 60/352,842, and 60/8352,841, all filed Feb. 1, 2002, of all of which are expressly incorporated by reference herein in their entirety.

### FIELD OF THE INVENTION

The present invention is directed to novel compositions and methods utilizing nanoparticles comprising protein cages and cores.

### BACKGROUND OF THE INVENTION

There is considerable interest in the controlled formation of size constrained and nanophase inorganic materials for a variety of technological applications such as magnetism, semiconductors [2, 3], ceramics [4] and medical diagnostics [5, 6, 7, 8]. However, conventional solution methods often produce materials having a range of particle sizes. Since the properties of nanophase materials are intimately related to their dimensions, this implies a heterogeneity of physical properties; this heterogenity limits their usefulness. Alternative syntheses using a biomimetic approach [9] have utilized organized molecular assemblies for materials synthesis, such as micelles, microemulsions, surfactant vesicles, Langmuir monolayers (and multilayers) and the protein cage of the iron storage protein ferritin. All these have proven to be versatile reaction environments and a wide range of inorganic materials have been synthesized using these systems [10]. However, there are severe limitations to these systems. Micelles for example are dynamic structures with fluctuations in size, whereas vesicles often have limited stability with regard to aggregation and hydrolysis. A major limitation to the existing synthetic methods, utilizing this biomimetic approach, has been the inability to vary particle size over a wide range while maintaining a narrow particle size distribution. The protein ferritin has provided a remarkably robust alternative for inorganic material synthesis [11]. Ferritins play a central role in the sequestration and storage of iron in biological systems. There is a high degree of structural conservation among ferritin proteins from different sources and all ferritins assemble, from multiple subunits, into a symmetrical cage-like structure. This protein cage acts to sequester Fe as a constrained nanoparticle of ferric oxyhydroxide (usually ferrihydrite). The reactions to form the mineral particle include the oxidation of Fe(II) and its subsequent hydrolytic polymerization to form the mineral. These reactions are catalyzed by the protein in two distinct ways. An enzymatic active site (ferroxidase) in the protein catalyzes the oxidation of Fe(II). The Fe(III) rapidly forms a small mineral core within the protein shell. This mineral surface will itself catalyze the oxidation of Fe(II) via the 4 electron reduction of O₂ to H₂O. Nucleation of the mineral particle inside the protein cage of ferritin occurs at symmetry related clusters of glutamic acid residues, which create a protein surface of high charge density. In the absence of the feroxidase site this highly charged interface is sufficient to induce oxidative hydrolysis and mineral formation within the confines of the protein cage.

Ultrafine particles are useful in the production of many materials ranging, for example, from coatings, particularly coatings of one or more layers, to high performance lubricants, and from electronic devices to therapeutic delivery systems. Traditionally, fine particles have been prepared by grinding larger particles. However, such grinding results in a heterogeneous mix of particle sizes and shapes, and thus limits the usefulness of such particles. Such mixes can be further fractionated, for example, by passage though one or more sieves. In this case, the fractions collected may be in a certain size range, but within that range the size and shape distribution remains heterogeneous. Moreover, this additional size selection may result in a large amount of material that is discarded. Due to the disparity in particle shapes and sizes, discontinuities, stresses, frictions, etc. may arise in the resultant material, layer, lubricant, etc. for which the particles are employed. Thus, even after the expenditure of much effort in the prior art, suitable particles for high performance and high tolerance applications could not heretofore be reliably and economically produced by grinding methods.

Attempts to circumvent these problems have met with limited success in the past. These alternative approaches have included condensation of vaporized atoms and controlled precipitation of solutes out of solutions. In the case of precipitation where seed particles are used, the heterogeneity of the seed particles themselves render mixtures that are polydisperse. There is thus a need in the art for monodisperse particles of a desired size and/or shape.

Bunker, et al ., “Ceramic Thin-Film Formation on Functionalized Interfaces Through Biomimetic Processing” Science 264: 48-55 (1994), discloses high density polycrystalline films of oxides, hydroxides and sulfides. These films are disclosed to be useful in a wide variety of applications. The films are prepared using substrates having functionalized surfaces. These surfaces are given a ceramic coating by the process of nucleation and particle growth mechanisms.

Aksay, et al., “Biomimetic Pathways for Assembling Inorganic Thin Films,” Science 273: 892 898 (1996) discloses a process whereby a supramolecular assembly of surfactant molecules at an organic-inorganic interface to template for condensation of an inorganic silica lattice. The technique is thought to be useful in the synthesis of inorganic composites with designed architecture at the nanometer scale.

Huo, et al., “Generalized Synthesis of Periodic Surfactant/Inorganic Composite Materials” Nature 368: 317-321 (1994), discloses the direct co-condensation of anionic inorganic species with cationic surfactants and the cooperative condensation of cationic inorganic species with anionic surfactants. The cooperative assembly of cationic inorganic species with cationic surfactants is also disclosed. The main driving force for this self-assembly is thought to be electrostatic. The technique is useful for synthesis of several different mesostructured phases.

Evans et al., “Biomembrane Templates for Nanoscale Conduits and Networks,” Science 273: 933-935 (1996) discloses the production of solid phase networks and conduits through the use of photochemical polymerization of long (20 to 200 nm) nanotubes. Nanotubes are formed by the mechanical retraction of a “feeder” vesicle after molecular bonding to a rigid substrate. Multiple nanotubes can be linked to form the networks and circuits.

Trau et al., “Field-induced Layering of Colloidal Crystals,” Science 272: 706709 (1996) discloses an electrohydrodynamic method for preparing a precise assembly of two- and three-dimensional colloidal crystals on electrode surfaces. The technique disclosed uses electrophoresis, with deposition and arrangement of the particles on the electrode. The technique provides for mono- or multi-layer crystalline films. It is also mentioned that the technique may be used to assemble macromolecules such as proteins into two dimensional crystals.

Monnier et al., “Cooperative Formation of Inorganic-Organic Interfaces in the Synthesis of Silicate Mesostructures,” Science 261: 1299-1303 (1993)) discusses a theoretical model of the formation and morphologies of surfactant silicate mesostructures. The article proposes a model for the transformation of a surfactant silicate system from the lamellar mesophase to the hexagonal mesophase. The effect of pH and ionic strength on mesophase structure are also discussed.

In a recent development, U.S. Pat. Nos. 5,304,382, 5,358,722, and 5,491,219, disclose the use of apoferritin devoid of ferrihydride as another solution to the problem of producing small particles. These ferritin analogs consist of an apoferritin shell and an inorganic core, and are thought to be useful in the production of ultrafine particles for high performance ceramics, drug delivery, and other uses.

Ferritin is a protein involved in the regulation of iron in biological systems. In nature, ferritin consists of a protein shell, having 24 structurally equivalent protein subunits surrounding a near spherical core of hydrous ferric oxide (“ferrihydrite”). The core is disclosed as being any organic or inorganic material with the exception of ferrihydrite. Once a core has formed in the process of these patents, the protein coat can be removed and the freed core particles isolated. The process is disclosed as providing for particles approximately 5 to 8 nanometers in diameter. However, this system is size constrained, such that homogeneous particles of smaller or larger sizes are not possible.

A general review of systems employing the apoferritin/core nanoscale particle production system is provided by Douglas, “Biomimetic Synthesis of Nanoscale Particles in Organized Protein Cages,” Biomimetic Materials Chemistry, S. Mann (ed.) VCH Publishers, New York (1996).

Additional information on the apoferritin/core system includes:

Douglas et al., “Inorganic-Protein Interactions in the Synthesis of a Ferromagnetic Nanocomposite,” American Chemical Society, ACS Symposium Series: Hybrid Organic-Inorganic Composites, J. Mark, C. Y-C Lee, P. A. Bianconi (eds.) (1995) discloses the preparation of a ferrimagnetic iron oxide-protein composite comprising an apoferritin shell and iron oxide core. The core is said to consist of magnetite or maghemite, but was thought to be predominantly maghemite. This magnetoferritin is said to be ideal for bio-compatible nmr imaging, and other biological and medical applications.

Douglas et al., “Synthesis and Structure of an Iron(III) Sulfide-Ferritin Bioinorganic Nanocomposite,” Science 269: 54-57 (1995) discloses production of iron sulfide cores inside ferritin shells via an in situ synthesis reaction. The cores are disclosed as a mostly amorphous sulfide consisting predominantly of Fe(III). Cores are described as a disordered array of edge-shared FeS.sub.2 units. Native ferritin particles with sulfided cores are taught to contain between 500 and 3000 iron atom cores, most predominantly in the Fe(III) form. Douglas et al. further disclose that the biomimetic approach to the production of nanoparticles may be useful for biological sensors, drug carriers, and diagnostic and bioactive agents.

Bulte et al., “Magnetoferritin: Characterization of a Novel Superparamagnetic MR Contrast Agent,” JMRI, May/June 1994, pp. 497-505, discloses use of horse spleen apoferritin to prepare nanoparticles having a ferritin shell and iron oxide core. The article discloses that novel materials with defined crystal size can be produced by “confined biomineralization within specific subunit compartments.” The magnetoferritin produced in the technique described is said to be useful in the production of a nanometer-scale contrast agent for magnetic resonance imaging. Coupling of “bioactive substances” to the ferritin case is further disclosed. Such substances are taught to include antibody fragments and synthetic peptides, which may be useful in tissue-specific imaging.

Meldrum et al., “Magnetoferritin: In Vitro Synthesis of a Novel Magnetic Protein,” Science 257: 522-523 (1992) discloses the preparation of magnetoferritin by incubation of apoferritin in a solution of Fe(II) and with slow oxidation. The process described resulted in the discrete, spherical nanometer (ca. 6.0 nm) core particles surrounded by a ferritin protein shell. The core was consistent with being either magnetite or maghemite, most likely magnetite. Possible uses for the magnetoferritin particles are disclosed as the following: (1) industrial applications, (2) study of magnetic behavior as a function of miniaturization, (3) elucidation of iron oxide biomineralization processes, (4) magnetic imaging of biological tissue, and (5) in separation procedures involving cell and antibody labeling.

Meldrum et al., “Reconstitution of Manganese Oxide Cores in Horse Spleen and Recombinant Ferritin,” Journal of Inorganic Biochemistry, 58: 59-68 (1995) discloses the formation of MnOOH cores within the nanoscale cavity of ferritin. Ferritin reconstitution with MnOOH cores is taught to be a nonspecific pathway, and an “all or nothing effect” (i.e., either unmineralized or fully loaded). Different apoferritin sources were used: (1) horse spleen ferritin, (2) recombinant H- and L-chain homopolymers and (3) H-chain variants containing site-directed modifications at the ferroxidase and putative Fe nucleation centers. The particle cores are described as being amorphous, whereas particles formed in bulk solution under substantially the same conditions were crystalline.

Bulte et al., “Initial Assessment of Magnetoferritin Biokinetics and Proton Relations Enhancement in Rats,” Acad. Radiol., 2: 871-878 (1995), discloses blood clearance, in vivo biodistribution and proton relaxation enhancement of magnetoferritin (1.4 mg Fe/kg) in nude rats carrying a xenografted human small cell lung carcinoma. The kinetics of blood clearance was biexponential with an initial half-life of 1.4 to 1.7 min and a longer component lasting several hours. Ex vivo relaxometry revealed uptake in the liver, spleen and lymph nodes when magnetoferritin was administered with or without a pre-injection of apoferritin. No involvement with ferritin receptors (displayed on the carcinoma) was seen. Magnetoferritin is said to be potentially useful as an imaging agent for liver, spleen and lymph nodes.

Bulte et al., “Magnetoferritin: Biomineralization as a Novel Molecular Approach in the Design of Iron-Oxide-Based Magnetic Resonance Contrast Agents,” Investigative Radiobiology 20 (Supplement 2): S214-S216 (1994) reports on the magnetometry and magnetic resonance relaxometry of magnetoferritin. Magnetoferritin is described as a biocompatible magnetic resonance contrast agent. The publication further discloses that magnetoferritin has a convenient matrix for complexing a wide variety of bioactive substances and may provide a basis for a novel generation of biocompatible magnetopharmaceuticals.

Accordingly, it is an object of the present invention to provide novel compositions and uses for nanoparticles comprising protein cages and guest molecule cores.

### SUMMARY OF THE INVENTION

In accordance with the objects outlined herein, the present invention provides compositions comprising protein cages, particularly dodecameric (12 subunit) protein cages loaded with at least one, and preferably a plurality (e.g. two or more) guest materials. Preferred embodiments utilize Listeria innocua ferritin-like protein cages, and in some aspects, includes the use of metal(s) as the guest molecules comprising the core. Particularly preferred metals that form nanoparticle cores include iron, iron oxides and mixtures of iron, cobalt, nickel and/or platinum, with other transition metals and particularly the lanthanides being preferred.

In a first aspect, the present invention provides solution phase nanoparticles and/or nanoparticle cores. In the former case, the nanoparticles may be derivatized with any number of molecules outlined herein, including biomolecules, dendrimers (including dopants, particularly metals). In additional aspects, the nanoparticles and/or nanoparticle cores (e.g. with the protein shells substantially removed or dissolved) are distributed on substrates, including in an ordered manner.

In an additional aspect, the invention provides solid supports comprising a plurality of first nanoparticles of a first size loaded with guest molecules, and a plurality of second nanoparticles of a second size similarly loaded. In some embodiments, the first and second nanoparticles are the same size. In some, the guest molecules are the same. Similarly, in some embodiments, two differently sized nanoparticles are used, with the same or different cores. Iron and iron mixtures are particularly preferred as core materials.

### DETAILED DESCRIPTION OF THE DRAWINGS

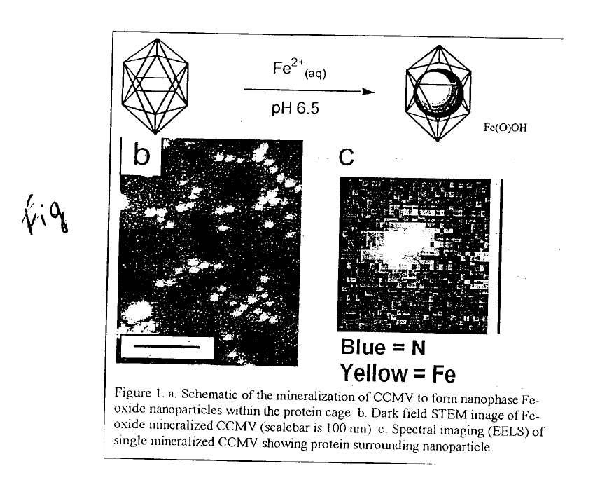

FIGS. 1A, 1B and 1C depict schematics associated with CCMV nanoparticles. 1A: Schematic of the mineralization of CCMV to form nanophase Fe-oxide nanoparticles within the protein cage 1B. Dark field STEM image of Fe-oxide mineralized CCMV (scalebar is 100 nm) 1C Spectral imaging (EELS) of single mineralized CCMV showing protein surrounding nanoparticle.

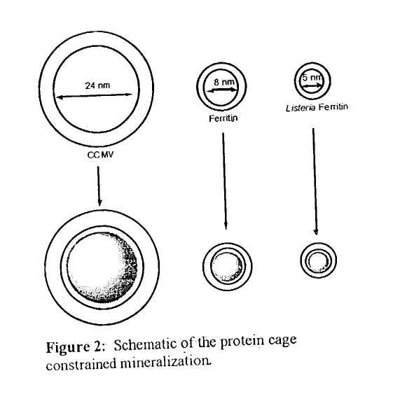

FIG. 2 is a schematic of the protein cage constrained mineralization.

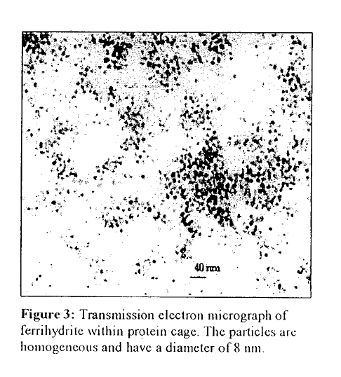

FIG. 3 is a transmission electron micrograph of ferrihydrite within protein cage. The particles are homogeneous and have a diameter of 8 nm.

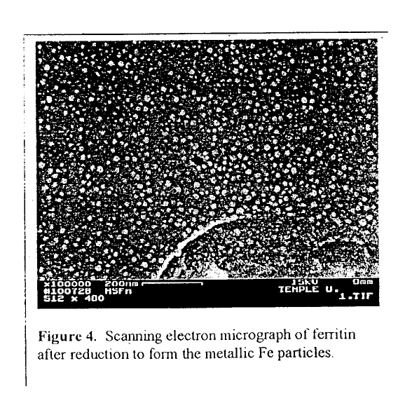

FIG. 4 is a scanning electron micrograph of ferritin after reduction to form the metallic Fe particles.

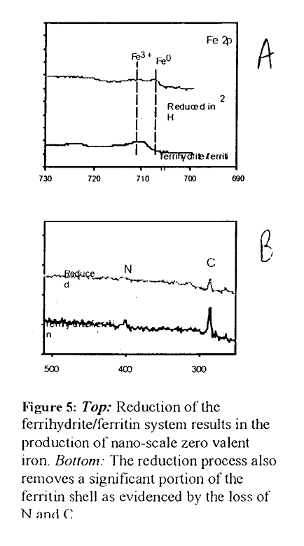

FIGS. 5A and 5B depict the production of nanoparticles of zero valent metals from oxide precursors. 5A: Reduction of the ferrihydrite/ferritin system results in the production of nano-scale zero valent iron. 5B The reduction process also removes a significant portion of the ferritin shell as evidenced by the loss of N and C.

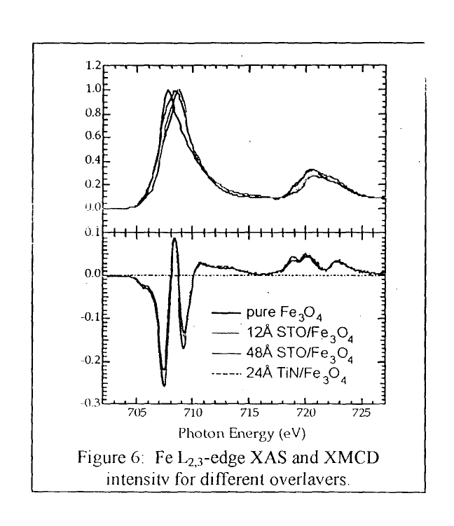

FIG. 6 FeL2,3-edge XAS and XMCD intensity for different overlayers.

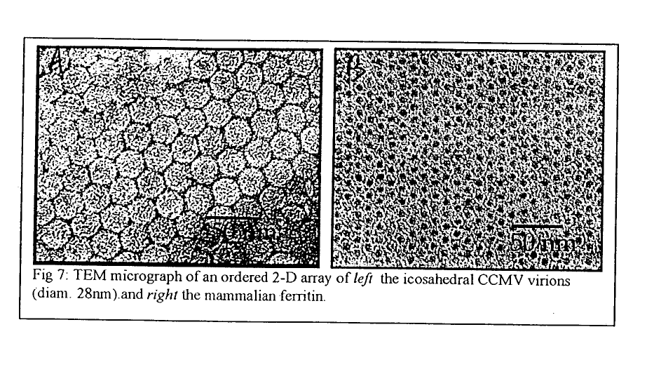

FIG. 7 depicts TEM micrographs of an ordered 2-D array of (A) the icosahedral CCMV virions (diameter 28 nm) and B the mammalian ferritin.

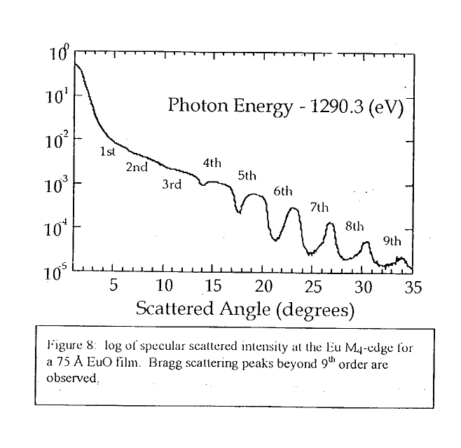

FIG. 8 log of specular scattered intensity at the Eu M4-edge for a 75 Å EuO film. Bragg scattering peaks beyond 9ₜₕ order are observed.

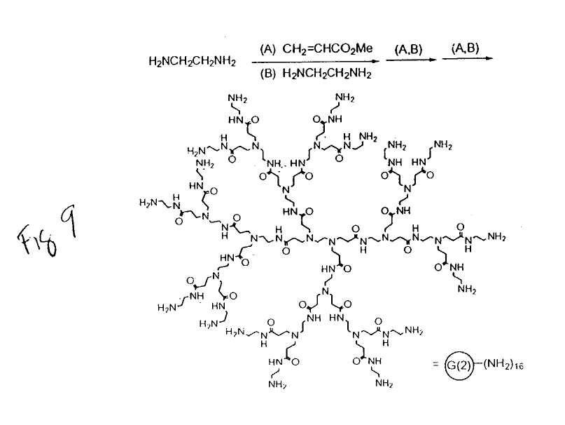

FIG. 9 depicts scheme 1 which is the synthesis of a second generation PAMAM dendrimer.

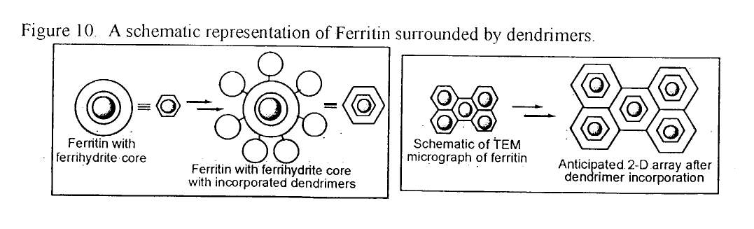

FIG. 10 depcits a schematic representation of ferritin surrounded by dendrimers.

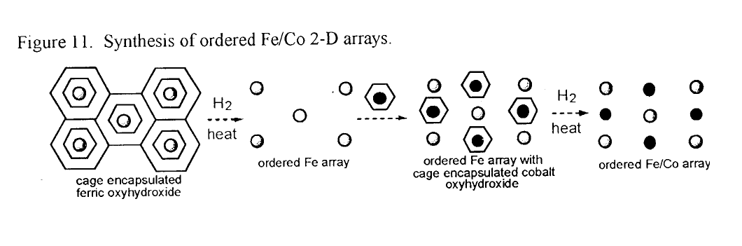

FIG. 11 depicts the synthesis of ordered Fe/Co 2D arrays using two different nanoparticles containing two different guest molecules. As will be appreciated by those in the art, while cores of pure Fe and pure Co are shown, mixtures of metals may be used for any particular core. IN addition, while FIG. 11 depicts the use of two different sized nanoparticles, one size with different cores may also be made.

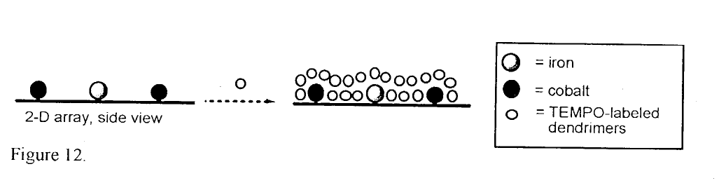

FIG. 12 depicts the side view of a nanoparticle array with dendrimers.

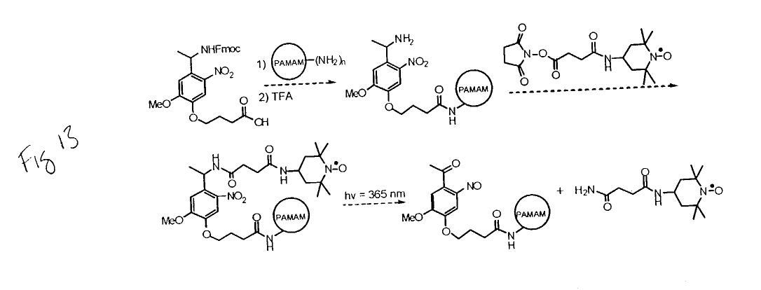

FIG. 13 depicts the derivatization of dendrimers (depicted as PAMAN dendrimers although other dendrimers can be used).

### DETAILED DESCRIPTION

The present invention is directed to the discovery that a variety of nanoparticles comprising protein cages can be made and mixed to produce materials with both a variety of new applications as well as “tunable” applications, e.g. the ability to alter material properties, e.g. different magnetic properties, by the incorporation of different elements in the nanoparticles and nanoparticle cores. This allows the directed synthesis of nanophase magnetic particulate materials whose magnetic properties are tailored by the size and composition of the particles, and by their assembly into mono- and multi-component two-dimensional ordered arrays. Thus, new magnetic materials are made whose component constituents are magnetic clusters that can be tightly tailored in size and magnetic composition, and whose mesoscopic magnetic properties (individual cluster moment, anisotropy, etc.) can be independently varied over a broad range. Furthermore, through the use of an appropriate interstitial material or derivatization of the shell materials, the assembly of these magnetic building blocks into ordered two-dimensional arrays allows for tunable and externally controllable inter-particle interactions that modify the macroscopic material properties for future application as superior performance magnetic memory, sensors, and ultra-high speed device architectures.

Previous work has utilized several different types of protein “shells” that can be loaded with different types of materials. For example, as outlined above and herein, virion nanoparticles comprising a shell of coat protein(s) that encapsulate a non-viral material have been described; see U.S. Pat. No. 6,180,389, hereby incorporated by reference in its entirety. Similarly, as described above and in references outlined in the bibliography, mammalian ferritin protein cages have been used that can be loaded with certain uniform materials.

The present invention is directed to the use of novel protein cages and mixtures of cages to form novel compositions, either in solution based systems and/or solid phase systems (e.g. two and three dimensional arrays on solid supports). The nanoparticles, which comprise both a protein “shell” and a “core”, can be mixed together to form novel compositions of either complete nanoparticle or core mixtures. In addition, the shells can be loaded to form the complete nanoparticles with any number of different materials, including organic, inorganic and metallorganic materials, and mixtures thereof. Particularly preferred embodiments utilize magnetic materials, to allow for high density storage capacities. Furthermore, as the shells are proteinaceous, they can be altered to alter any number of physical or chemical properties by a variety of methods, including but not limited to covalent and non-covalent derivatization as well as recombinant methods.

One of the advantages of the present invention is to enable the introduction or synthesis and encapsulation of nanoparticles, which cannot be accomplished through techniques and means disclosed in prior art. Another substantial advantage over prior art is the ability to vary the size of the nanoparticle encapsulated and constrained in the protein cage structure. It should be easily recognized that a portion of the volume within a given cage structure will be filled with ferrihydrite (in the case of ferritin structures) and thus a smaller nanoparticle of, for example CoPt, could be encapsulated compared to said nanoparticle encapsulated in an identical apoferritin structure. Furthermore, in the synthesis of iron containing molecules and structures, for example FePt, one can utilize the ferrihydrite present as one of the starting materials. Another advantage of the present invention is to improve the efficiency for encapsulation of nanoparticles by eliminating processing steps—compared to both apoferritin methods taught by the prior art.

The present invention also serves to enhance the usefulness of the encapsulated and constrained nanoparticles of the present invention by modification of the surfaces and interfaces of the protein cage structure. It is known in the prior art that various modifications to the outside of ferritins can be accomplished through chemical, physical and/or gene modification technology. These modifications can enable or prohibit attachment of the ferritin or other protein cage structures to other similar structures, can provide a means to bind to targets of interest for medical applications, can provide a means and method of fabricating two and three dimensional arrays of like, similar or different combinations of nanoparticles constrained by ferritin and other protein cage structures.

In the formation of useful arrays of nanoparticles, an essential element is a matrix of material surrounding and joining the nanoparticles, which may be insulating, semiconducting, or conducting. It is an object of this invention to chemically, genetically or physically modify the outside of the protein cages to enable self-assembly of arrays through the utilization of other organic or inorganic materials. This invention discloses the use of a matrix material, which could be self-assembled, that utilizes ferritins, other proteins and other organic macromolecules to fill the interstices between the nanoparticles. Ferritin cages identical to those forming the primary array of nanoparticles but that contain nanoparticles having other desired properties can also be used. The use of identical protein cages containing insulating or semiconducting materials as the intersticial materials could be particularly advantageous.

The present invention also enhances the usefulness of the constrained nanoparticles by modification of the interfaces through chemical or other means as disclosed in prior art to enable opening and closing the structure for introduction or extraction of the materials contained therein.

Thus, the present invention enhances the usefulness of the constrained nanoparticles by employing specific combinations of constrained nanoparticles, surface modification and interface modification to enable specific desired outcomes. For example a FePt core may be constrained within a ferritin cage and through appropriate surface modification arrays can be formed into two-dimensional arrays for use in floating gate magnetic memory applications. Techniques for burning away or otherwise eliminating the protein structure to produce a uniform array of cores are well known in the art and described below.

The encapsulated or constrained nanoparticles and/or nanoparticle cores of the present invention have many utilities including drug delivery, catalysis, semiconductor technology, ultra-high density recording, nanoscale electronics, and permanent magnets.

Accordingly, the present invention provides compositions comprising a plurality of nanoparticles. By “nanoparticle” herein is meant a composition of a proteinaceous shell that self-assembles to form a protein cage (e.g. a structure with an interior cavity which is either naturally accessible to the solvent or can be made to be so by altering solvent concentration, pH, equilibria ratios, etc.), which cage has been loaded with a material as discussed below. That is, a “nanoparticle” includes both the shell (e.g. protein cage) and the nanoparticle core. As outlined herein, different protein cages lead to different sized cores. Preferred embodiments utilize cores ranging from 1 to 30 nm (e.g. the internal diameter of the shells) with from about 5 to 24 nm being preferred (representing 8.5 to 28 nm outer shell diameters, in general, particularly when non-viral protein cages are used. Preferred non-viral protein cages include ferritins and apoferritins, both eukaryotic and prokaryotic species, in particular mammalian and bacteria, with 12 and 24 subunit ferritins being especially preferred. In addition, 24 subunit heat shock proteins forming an internal core space are included.

Mammalian ferritin is a metalloprotein complex formed from a roughly spherical core containing about 3,000 inorganic atoms such as iron, and a shell of 24 identical subunits each having a molecular weight of about 20 kD. The outer diameter of mammalian ferritin is roughly 12 nm and the core is roughly 8 nm. Ferritin without the iron core molecules is called apoferritin. Listeria innocua has a ferritin-like structure that catalyzes the oxidation of Fe(II) and is a dodecameric (12 subunits, rather than 24) protein. There are a variety of other self-assembling “shells” known, including the dodecameric Dsp heat shock protein of E. coli and the MrgA protein as well as others known in the art. As will be similarly appreciated by those in the art, the monomers of the protein cages can be naturally occurring or variant forms, including amino acid substitutions, insertions and deletions (e.g. fragments) that can be made for a variety of reasons as further outlined below. For example, amino acid residues on the outer surface of one or more of the monomers can be altered to facilitate functionalization for attachment to additional moieties (targeting moieties such as antibodies, polymers for delivery, the formation of non-covalent chimeras), to allow for crosslinking (e.g. the incorporation of cysteine residues to form disulfides). Similarly, amino acid residues on the internal surfaces of the shell can be altered to facilitate guest molecule loading, stability, to create functional groups which may be later modified by the chemical attachment of other materials (small molecules, polymers, proteins, etc.).

In a preferred embodiment, in particular with the dodecameric protein cages, the natural channels to the interior formed by the the two-, three-, and four-fold symmetry of the dodecameric proteins may be modified to enable either the introduction and/or extraction, or both, of materials through the opening therein.

In preferred embodiments, covalent modifications of protein cages are included within the scope of this invention. One type of covalent modification includes reacting targeted amino acid residues of cage residue with an organic derivatizing agent that is capable of reacting with selected side chains or the N-or C-terminal residues of a cage polypeptide. Derivatization with bifunctional agents is useful, for instance, for crosslinking the cage to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3[(p-azidophenyl)dithio]propioimidate. Crosslinking agents find particular use in 2 dimensional array embodiments.

Alternatively, functional groups may be added to the protein cage for subsequent attachment to additional moieties. Preferred functional groups for attachment are amino groups, carboxy groups, oxo groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, as well as the 2003 catalog, both of which are incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of cages, if appropriate, comprises altering the native glycosylation pattern of the polypeptide. “Altering the native glycosylation pattern” is intended for purposes herein to mean deleting one or more carbohydrate moieties found in native sequence of the cage monomer, and/or adding one or more glycosylation sites that are not present in the native sequence.

Addition of glycosylation sites to cage polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of cage moieties comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337. This finds particular use in increasing the physiological half-life of the composition.

Cage polypeptides of the present invention may also be modified in a way to form chimeric molecules comprising an cage polypeptide fused to another, heterologous polypeptide or amino acid sequence. In one embodiment, such a chimeric molecule comprises a fusion of a cage polypeptide with a tag polypeptide which provides an epitope to which an anti-tag antibody can selectively bind. The epitope tag is generally placed at the amino-or carboxyl-terminus of the polypeptide. The presence of such epitope-tagged forms of a cage polypeptide can be detected using an antibody against the tag polypeptide. Also, provision of the epitope tag enables the cage polypeptide to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In a preferred embodiment, the nanoparticles are derivatized for attachment to a variety of moieties, including but not limited to, dendrimer structures, additional proteins, carbohydrates, lipids, targeting moieities, etc. In general, one or more of the subunits is modified on an external surface to contain additional moieties.

In a preferred embodiment, the nanoparticles can be derivatized as outlined herein for attachment to polymers. The character of the polymer will vary, but what is important is that the polymer either contain or can be modified to contain functional groups for the attachment of the nanoparticles of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Particularly preferred polycations are polylysine and spermidine. Both optical isomers of polylysine can be used. The D isomer has the advantage of having long-term resistance to cellular proteases. The L isomer has the advantage of being more rapidly cleared from an animal when administered. As will be appreciated by those in the art, linear and branched polymers may be used.

A preferred polymer is polylysine, as the —NH₂ groups of the lysine side chains at high pH serve as strong nucleophiles for multiple attachment of nanoparticles. At high pH the lysine monomers can be coupled to the nanoparticles under conditions that yield on average 5-20% monomer substitution.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, a preferred size for the polymer is from about 10 to about 50,000 monomer units, with from about 2000 to about 5000 being particularly preferred, and from about 3 to about 25 being especially preferred.

In a preferred embodiment, a targeting moiety is added to the composition. It should be noted that in the case of polymers, the targeting moiety may be added either to the nanoparticle itself or to the polymer. By “targeting moiety” herein is meant a functional group which serves to target or direct the complex to a particular location, cell type, diseased tissue, or association. In general, the targeting moiety is directed against a target molecule and allows concentration of the compositions in a particular localization within a patient. In a preferred embodiment, the agent is partitioned to the location in a non-1:1 ratio. Thus, for example, antibodies, cell surface receptor ligands and hormones, lipids, sugars and dextrans, alcohols, bile acids, fatty acids, amino acids, peptides and nucleic acids may all be attached to localize or target the nanoparticle compositions to a particular site.

In a preferred embodiment, the targeting moiety allows targeting of the nanoparticle compositions to a particular tissue or the surface of a cell.

In a preferred embodiment, the targeting moiety is a peptide. For example, chemotactic peptides have been used to image tissue injury and inflammation, particularly by bacterial infection; see WO 97/14443, hereby expressly incorporated by reference in its entirety.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin 5 heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells, including, but not limited to, HER2.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In one embodiment, antibodies against virus or bacteria can be used as targeting moieties. As will be appreciated by those in the art, antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like) may be used.

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

As outlined herein, targeting moieties can be organic species including biomolecules are defined herein. In a preferred embodiment, the targeting moiety may be used to either allow the internalization of the nanoparticle composition to the cell cytoplasm or localize it to a particular cellular compartment, such as the nucleus.

In a preferred embodiment, the targeting. moiety is all or a portion of the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); Baldin et al., EMBO J. 9:1511 (1990); Watson et al., Biochem. Pharmcol. 58:1521 (1999), all of which are incorporated by reference.

In a preferred embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFκB p50 (EEVQRKRQKL; Ghosh et al., Cell 62:1019 (1990); NFκB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In a preferred embodiment, targeting moieties for the hepatobiliary system are used; see U.S. Pat. Nos. 5,573,752 and 5,582,814, both of which are hereby incorporated by reference in their entirety.

Other modifications include the addition of dendrimers to the interstitial space of the cage, further outlined below.

In general, the protein cages are made recombinantly and self assemble upon contact (or by alteration of their chemical environment). As will be appreciated by those in the art, there are a wide variety of available techniques for the production of proteins in a wide variety of organisms.

In addition to protein cages, some embodiments of the invention, for example those utilizing arrays and compositions of mixtures of nanoparticles and nanoparticle cores, the nanoparticles can utilize viral protein cages, such as those of the CCMV virus as well as others, including tobacco mosaic virus (TMV); see U.S. Pat. No. 6,180,389, hereby incorporated by reference in its entirety. TMV serves as a particularly good “spacer” given its size.

The protein cages are loaded with materials. By “loaded” or “loading” or grammatical equivalents herein is meant the introduction of non-native materials (sometimes referred to herein as “guest molecules”) into the interior of the protein shell (sometimes referred to herein as “mineralization”, depending on the material loaded). In preferred embodiments, the protein shells are devoid of their normal cores; e.g. ferritins in the absence of iron (e.g. apoferritins) are loaded; alternatively, additional loading is done in the presence of some or all of the naturally occurring loading material (if any). In general, there are two ways to control the size of the core; by altering the cage size, as outlined herein, or by controlling the material to protein shell ratio (e.g. the loading factor). That is, by controlling the amount of available material as a function of the amount and size of shells to be loaded, the loading factor of each individual particle can be adjusted. For example, as outlined below, mammalian ferritin shells can generally accommodate as many as 4,000 iron atoms, while Listeria shells can accommodate 500. These presumably maximum numbers may be decreased by decreasing the load factors. In general, the loading is an equilibrium driven passive event or entrapment (although as outlined below, the natural channels or “holes” in the shells can be manipulated to alter these parameters), with physiological buffers, temperature and pH being preferred, with loading times of 12-24 hours. Typically, for the mineralization of the 24 subunit ferritin, aliquots of Fe2+ (25 mM as (NH₄)Fe(SO4)2.6H2O) are added to a solution of apoferritin (1 mg) in roughly 4 mL of a morpholine sulfonate buffer (MES (0.1 M, pH 6.5) and stirred with a magnetic stirrer. The Fe(II) is added in aliquots of 40 μL corresponding to ˜500 Fe2+ atoms/protein cage. The reaction to stir and air oxidize for ˜1 hour between additions and left to stir overnight (±24 hrs) at 4° C. The same procedure is used for mineralizing the other cages (Listeri ferritin-like protein and CCMV) but with slightly different amounts of Fe. Other buffers can be used and the pH of the reaction can be altered between 6 and 9. We can also do the reaction in the absence of any buffer and changes in pH can be titrated using an auto-titrator. In addition, these general conditions work for other metals as well. Preferred embodiments generally utilize solutions of anywhere from 10000:1 to 1:1 material:shell.

The protein cages are loaded with materials. In this context, “material” includes both inorganic, organic and organometallic materials, ranging from single atoms and/or molecules to large conglomerates of the same.

In a preferred embodiment, the protein cages are loaded with inorganic materials, including, but not limited to, metals, metal salts, metal oxides (including neat, doped and alloyed metal oxides), non-metal oxides, metal and non-metal chalcogens, sulfides, selinides, coordination compounds, organometallic species. Suitable metals include, but are not limited to, monovalent and polyvalent metals in any form depending on the end use of the nanoparticle and/or core; e.g. elemental, alloy (where relative concentrations of the elements can vary continuously—(Co/Ni, Co/Fe/Ni etc.)) and intermetallic (which are distinct compounds with definite stoichiometries—(CO₃Pt, FePt, FePt₃ etc.)). For monovalent metal salts, silver chloride may be used to nanoparticles useful in photographic applications. Polyvalent metals include, but are not limited to, transition metals and mixtures, including aluminum, barium, chromium, cobalt, copper, europium, gadolinium, lanthanum, magnesium, manganese, nickel, platinum, neodymium, titanium, yttrium, zirconium, terbium, zinc and iron, as well as other lanthanides. Metals that can possess magnetic properties such as iron are particularly preferred. Preferred embodiments utilize mixtures of metals, such as Co, Ni, Fe, Pt, etc. as outlined herein.

In a preferred embodiment, as outlined in the examples, the nanoparticles can be made with zero valent metals from oxide precursors. In this embodiment, the shells are loaded with metal oxides such as iron oxide and then reduced using standard techniques.

In addition, the material may be any number of organic species, including but not limited to organic molecules and salts thereof, with biomolecules being particularly preferred, including, but not limited to, proteins, nucleic acids, lipids, carbohydrates, and small molecule materials, such as drugs, specifically including hormones, cytokines, antibodies, cellular membrane antigens and receptors (neural, hormonal, nutrient, and cell surface receptors) or their ligands, etc). The present invention finds particular use in the delivery of therapeutic moieties to organisms, including tissues and cells; for example, the shell component of the nanoparticle can serve as a type of “controlled release” delivery system. As will be appreciated by those in the art, any number of suitable drugs such as those found in the Physician's Desk Reference can be used. In addition, as further described below, the moieties defined below as suitable guest molecules may also serve as “targeting moieties” when attached to the surface of the shell and/or nanoparticle.

By “nucleic acid” or “oligonucleotide” or grammatical equivalents herein means at least two nucleotides covalently linked together. A nucleic acid of the present invention will generally contain phosphodiester bonds, although in some cases, for example when therapeutic antisense molecules are to be included in the nanoparticle core, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, depending on its ultimate use, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration.

By “carbohydrate” herein is meant a compound with the general formula Cx(H₂O)y. Monosaccharides, disaccharides, and oligo- or polysaccharides are all included within the definition and comprise polymers of various sugar molecules linked via glycosidic linkages. Particularly preferred carbohydrates (particularly in the case of targeting moieties, described below) are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates.

“Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

As will be appreciated by those in the art, the compositions of the invention can include a wide variety of different mixtures of “shells” and “cores”, with mixed compositions being preferred, and matrices of different sized nanoparticles and/or cores with different core compositions being possible, as outlined herein.

In a preferred embodiment, the compositions comprise a solid support that contain the nanoparticles of the invention. By “substrate” or “solid support” or other grammatical equivalents herein is meant any material that can be modified to contain discrete individual sites appropriate for the attachment or association of beads and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc.

Generally the substrate is flat (planar), although as will be appreciated by those in the art, other configurations of substrates may be used as well; for example, three dimensional configurations can be used, for example by using previously micromachining or semiconductor manufacturing methods to create fine structures onto which the nanoparticles are to be deposited.

The nanoparticles are generally distributed on the substrate via contacting the two in any number of ways. In a preferred embodiment, assembly can be accomplished by a) spin coating using a solution containing the protein b) through monolayer formation at an air-water interface (Langmuir monolayer) and subsequent transfer to the substrate of interest c) Formation of actived (acivatable) self assembled monolayers on Ag, Au, Si, SiO2 surfaces followed by adsorbtion of the proteins onto those surfaces. This will include making SAMs that are terminated with amines (cationic), sulfates, sulfonates, carboxylates, phosphonates etc (anionic), also activated heagroups such as succinimidyl esters, maleimides. Other means for formation of protein arrays involves the modification of the protein cage to introduce reactive thiol (SH) groups on the exterior surfaces of the protein (done either genetically or chemically) and subsequent adsobtion of the protein directly onto a Au or Ag surface.

In addition to chemical functionalization of the surface for absorption or covalent attachment, other associative techniques may be used, for example through the use of adhesives (see for example U.S. Pat. No. 6,143,374, hereby expressly incorporated by reference.

When the nanoparticles or the nanoparticle cores are used in an array format (e.g. on a solid support), the interstitial spaces between the proteins forming the cage can be modified to include additional materials, termed herein “spacer materials”, including insulating, semiconductive and conductive materials, magnetically inert materials, etc.

In a preferred embodiment, the spacer material comprises dendrimers. As will be appreciated by those in the art, a variety of dendrimeric structures find use in the present invention, in general any dendrimer that can incorporate dopants that allow for the alteration of magnetical and electrical properties as is known in the art can be used.

In a preferred embodiment, the spacer material comprises an insulating material as is known in the art, including Organic polymer, SiO₂, Al₂O₃, and any number of well known additives.

Once made, the compositions of the invention find use in a variety of applications. In general, methods, nanoparticles, and arrays, according to the present invention provide a means to generate magnetic materials comprising magnetic clusters of specifically designed size and magnetic composition, and whose mesoscopic magnetic properties may be independently varied over a large range. That is, by choosing the type and size of nanoparticle at each, or at least a plurality of, locations within the magnetic material, or a specific magnetic cluster, the individual cluster moment (dipole), and anisotropy (or tendency of the material to magnetized), as well as other properties, may independently designed and controlled. Further, through the appropriate choice of interstitial materials and the formation of arrays, inter-particle interactions are controlled, allowing for specific design of macroscopic magnetic material properties, such as the coercive field. This overall design capability—that is, the ability to independently vary individual magnetic properties, and/or the ability to design a magnetic material by choosing the size and type of a plurality of nanoparticles that make up the material, as well as the interstitial molecules that govern one or more interparticle interactions—allows for the design of unprecedented magnetic materials. In this general manner, methods, nanoparticles, and arrays of the present invention find use in generally any present or future application requiring or advantageously employing a magnetic material or device, in that the methods, nanoparticles, and arrays of the present invention allow for the precise and independent tailoring of magnetic materials for any application.

Two-dimensional arrays of nanoparticles according to the present invention may be used in magnetic memory applications, including but not limited to floating gate magnetic memories. Methods according to the present invention for providing nanoparticles having a diameter of less than 6 nm, finds use in the formation of magnetic media incorporating the nanoparticles. Magnetic media incorporating the small diameter nanoparticles taught by the present invention has an increased density of magnetic particles than media found in the prior art, and therefore an increased storage density.

Further, the present invention provides nano-particles of substantially spherical particles of 5 nm in diameter with little variation in size. That is, relative to methods taught in the prior art, methods according to the present invention provide nanoparticles having a predictable diameter as provided herein. The inventive method provides nanoparticles having a diameter as outlined below, with remarkable reproducibility of size. The production of nanoparticles with a narrow width distribution finds use in forming finely textured arrays of magnetic particles for use, for example, in forming higher density magnetic storage devices. In an analogous manner, finer recording heads may be fabricated, allowing for higher density magnetic storage to be achieved.

Accordingly, particles and arrays according to embodiments of the present invention will find use in magnetic memories and media having increased speed, access, density, reduced power consumption, and reduced weight.

The small nanoparticles formed according to embodiments of the present invention further find use in enhancing the rate and specificity of various reactions—including catalytic and stoichiometric reactions. Particles having smaller diameters, such as the 6 nm nanoparticles made according to embodiments of the present invention, enhance reaction rates of certain reactions due to their greater surface area-to-mass ratio. Accordingly, nanoparticles according to embodiments of the present invention find use in a variety of industries and applications including petroleum refining, chemical production, foods, medicines, drug delivery, catalysis, environmental remediation, chemical and biological sensors, lubricants, coatings, separation media, photo-activated reactions, semiconductor technology, ultra-high density recording, nanoscale electronics, and permanent magnets.

In a preferred embodiment, the arrays (and solutions) comprising the nanoparticles, particularly the nanoparticle cores, find use as metal catalysts.

One application provided herein is the use of the solid phase arrays for processing fine structures, for example in the semiconductor device area. In this embodiment, similar to the process described in U.S. Patent Application 2002/0192968, the nanoparticles comprising inorganic cores are arranged on a support and the organiFc shells are removed, leaving the inorganic cores on the surface to serve as an etching mask.

In a preferred embodiment, the compositions of the invention are used to deliver therapeutic moieties to patients. The administration of the compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the composition may be directly applied as a solution or spray. Depending upon the manner of introduction, the nanoparticles may be formulated in a variety of ways, including as polymers, etc. The concentration of therapeutically active compound in the formulation may vary from about 0.1-100 wt. %.

The pharmaceutical compositions of the present invention comprise nanoparticles loaded with therapeutic moieties in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of the compositions may be administered. Moreover, the compositions may be administered in combination with other therapeutics.

Generally, sterile aqueous solutions of the nanoparticles of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred.

In some embodiments, it may be desirable to increase the blood clearance times (or half-life) of the nanoparticle compositions of the invention. This has been done, for example, by adding carbohydrate polymers, including polyethylene glycol, to other compositions as is known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

While mammalian ferritins are comprised of 24 structurally similar polypeptide subunits that self-assemble to form a protein cage structure, the ferritin from the bacteria Listeria innocua assembles into a cage like structure having only 12 subunits. The outside diameter of the mammalian ferritin is 12 nm while that of the Listeria ferritin is only 8.5 nm. Up to 4000 Fe atoms can be mineralized and stored within the mammalian ferritin cage as a nanoparticle of ferric oxyhydroxide (ferrihydrite, Fe(O)OH) while only 500 Fe atoms can be stored in the Listeria ferritin cage. As detailed in later sections, we have successfully produced ferritin cages of both size classes giving us a powerful set of varying sized ferritin cages.

We have also demonstrated that virus particles devoid of their nucleic acid can be utilized as novel size constrained reaction vessels for material synthesis ([12]; FIG. 1) thereby extending the range of synthetic protein cages. We have used the assembled protein shell of Cowpea chlorotic mottle virus (CCMV), a plant virus to initiate and constrain crystallization to form nanophase particles of metal-oxide mineral phases [13-15]. The size homogeneity of the material is a consequence of the discrete virus dimensions, while the mineralization (loading) process appears to be controlled by specific inorganic-organic interactions at the interior surface of the virus particle. This robust cage can be genetically engineered to alter its chemical characteristics to direct a broad range of nano-materials synthesis. We have extensively developed the CCMV in vitro assembly system to understand, in chemical detail, the interactions that dictate virion assembly, stability, and disassembly [16-20].]. This allows us to assemble protein cages comprising different modified subunits for the multivalent presentation of functional groups/ligands, targeting agents on the exterior and interior surfaces of the protein cages. This detailed work makes the CCMV system of particular importance for nanoparticle synthesis: high stability cage structures in a diversity of shapes and sizes have been assembled from the CCMV subunit protein. For example, CCMV protein cages comprised of 60 subunits (diam 12 nm) or 120 subunit (diam 22 nm), as well as the 180 subunit (28 nm diameter) have been produced using genetic modifications of the CCMV subunits. This demonstrates the robustness of the viral subunits to produce a wide range of cage structures from a common subunit.

The unique benefit of the biomimetic approach of using protein cages as constrained reaction environments presented in this proposal lies in the diversity of protein cage sizes and structural stability with the potential to act as constrained reaction environments for nanomaterial synthesis.

In the nanoparticles that we are synthesizing, the magnetic properties are primarily influenced by core composition and cage size. In two-dimensional arrays of the particles, additional interactions are introduced that lead to newly emergent magnetic properties. Inter-particle interactions can be tailored by the use of different chemical crosslinks, in particular crosslinks bearing spins. Ordered arrays made from more than one particle type will yield a new type of ordered magnetic alloy. Ordered alloys (of atoms) have played in important role in magnetic phenomena. With the additional long-range periodicity, major changes in the electronic and magnetic properties of the material occur. Alloy ordering is often the progenitor of additional periodicities (beyond spin ordering like ferromagnetism) including orbital ordering, Brillouin zone folding, formation of charge density waves and spin density waves, Pieris instabilities, and Jahn-Teller distortions. It is also responsible for technologically superior properties such as enhanced Kerr rotation in magneto-optical recording media and sensors, increased magnetic anisotropy to over superparamagnetic limits in ultra-dense recording media, and substantially improved M-H energy products in hard magnet systems. Beyond the systematic understanding of coupling mechanisms, the utility of independent variability of the coupling strength (via modification of the interstitial media) is the tailored control of macroscopic material parameters. Again, in analogy to bulk magnetism, modification of the spin-spin coupling strength is the driving force for both long range magnetic order and the global magnetic properties (Kerr rotation, magnetic anisotropy energies, spin injection efficiencies). Tailoring and externally controlling the inter-particle coupling strength is a tremendously beneficial effect.

We have developed a unique set of tools with which to create and analyze novel ordered magnetic arrays. These include 1) a set of protein cages of different sizes that we have demonstrated can act as constrained reaction environments; 2) the ability to reduce transition metal oxide nanomaterials to their corresponding metallic nanoparticles; 3) demonstrated ability to fabricate ordered 2-d arrays of these protein cages; and 4) synthesis of organic spin labeled materials to mediate coupling between metallic nanoparticles. By using our established synthesis of protein constrained metal oxide nanoparticles of controlled size and composition, we fabricate ordered 2-D arrays of magnetic nanomaterials. From the patterned arrays of protein encapsulated oxide materials we can derive zero valence metal nanoparticles by reduction. We can presently synthesize size-constrained nanoparticle cores of iron oxide and cobalt oxide within protein cages ranging in size from 8 nm to 28 nm diameter. These protein encapsulated nanoparticle cores can be easily fabricated into ordered 2-D arrays. Furthermore, preliminary results demonstrate our ability to reduce the iron oxide nano-particles to yield nano-sized zero valent iron metal particles. At least one prior impediment to fully investigating and ultimately testing the utility of nano-structures has been the difficulty of preparing mondispersed nanomaterials. Our bioengineering approach addresses and circumvents this difficulty. In addition, the use of proteins as size constrained reaction vessels has an additional advantage in that these materials can be easily fabricated into ordered 2-D arrays on a variety of substrates. Introduction of small organic based particles into the 2-D arrays allows for controlled modulation of the properties of the magnetic composites. Timely characterization by magnetometry and polarized X-ray absorption spectroscopy and X-ray scattering will serve to both correct and confirm our synthesis capabilities.

Production of Protein Cage Structures:

Robust expression and purification systems have been developed for the three protein cages utilized in this proposal (ferritin and viral based cages). These expression systems allow for the routine production of milligram to gram quantities for each of the protein cages. We have cloned and expressed both the mammalian and Listeria ferritins in E. coli using the pET-based expression vectors. The purified ferritins from E. coli self assemble into structures identical to the native protein cages. The CCMV coat protein has been cloned and expressed using the Pichia pastoris system. The coat protein self assembles within P. pastoris into an empty 28 nm protein cage indentical in size to the native virus. Fermentation technology has been developed for the expression of the viral protein cage. We can produce significant quantities of these cages and their corresponding nanoparticles, including a variety of different cores.

Synthesis of size constrained, homogeneous nano-particles using protein cages We have demonstrated biomimetic mineralization of transition metal oxides in the three unique self-assembled protein cages, 24 nm, 8 nm and 5 nm.

We have used the ferritin cages to synthese a range of transition metal oxide nanoparticles. These include the synthesis of magnetic iron oxide nanoparticles, 8 nm and 5 nm in diameter, in the mammalian and Listeria ferritins (FIG. 3.) respectively. These materials are both compositionally homogeneous and monodisperse and provide one of the best available synthetic approaches to nano-particle synthesis. In addition, we have used both the mammalian ferritin and Listeria ferritin to synthesize monodisperse 8 and 5 nm diameter particles of Co-oxyhydroxide. Therefore, we have demonstrated that the required starting materials for the formation of our proposed magnetic materials are in hand.

We have previously shown that native virus protein cage of CCMV can be used for the synthesis of polyoxometalate nanoparticles (21). In addition, we have recently shown that the specific mineralization chemistry of ferritin can be genetically engineered into these viral protein cage architectures (22) which can be used to synthesize 24 nm diameter iron oxide nanoparticles. Thus, we have shown that the viral protein cage of cowpea chlorotic mottle virus (CCMV) will encapsulate and size-constrain inorganic nanoparticles based on electrostatic interactions on the interior of the viral protein cage. This is useful as a model for mineralization in ferritin but also provides an additional size dimension for our synthetic arsenal of size-constrained reaction vessels. Initial experiments with the engineered viral protein cage suggest that Co-oxyhydroxide mineralization occurs in a similar manner to the ferritins described above. Therefore, we have at our disposal three protein cage systems all of which are capable of mineralizing and encapsulating ferric- and cobalt-oxyhydroxide nanoparticles.

In addition, we can control particle size, within any of these protein cages, by adjusting the reaction conditions, in particular the ratio of metal ion to protein (23). There are two approaches to controlling the size of the metal oxide nano-particle a) using a protein cage structure of appropriate size from our library of active cage structures (CCMV, ferritin, Listeria ferritin) b) controlling the to protein ratio (loading factor). Therefore, particles with diameters ranging from 2 to 24 nm can be synthesized within the protein cages.

Compositional variation of protein encapsulated nano-particles The composition of the inorganic nanoparticles encapsulated within the protein cages can be manipulated to produce a range of transition metal oxide particles (21,22). For example we have shown that in the presence of mammalian ferritin, oxidation of Co(II) will lead to the mineralization of a cobalt oxyhydroxide Co(O)OH, 8 nm in diameter, constrained by the protein cage. This result has been duplicated with the Listeria ferritin which undergoes an almost identical reaction to produce 5 nm diam. particles of Co(O)OH within the protein cage. In addition we have made mixed Co—Fe oxide nanoparticles within the mammalian ferritin cage and while this material has not been fully characterized it does show a consistent spectral shift with composition and the magnetic properties are consistent with a compositionally mixed Fe—Co oxide. Fe- and Co-oxyhydroxide minerals can be introduced into the protein cages under identical conditions (pH, temperature) and the reactions proceed via the slow addition of the divalent metal ion and an oxidant. Therefore, by varying the ratio of metal ions in the reaction, nanoparticles of variable composition (CoxFey(O)OH) have been achieved using mammalian ferritin. By changing the oxidant the valence of the metal ion in the final mineral can be controlled i.e. O2 will oxidize Fe(II) but not Co(II) whereas H2O2 will oxidize both. Using this approach it is possible to also dope in a certain amount of other metal ions such as Zn2+, Eu2+/3+, and Ni2+ which might not undergo the same oxidative-hydrolysis chemistry, as Co and Fe, to form a mineral solid. The significance of this is that we can control and characterize the compositional alloys of metal oxide nanoparticles. Also, we can do synthesize these materials within our set of differently sized protein cages giving us both size control in addition to compositional control.

Nanometallic Materials

Production of Nano Particles of Zero Valent Metals From Oxide Precursors

An exciting recent development has been our ability to synthesize iron oxide particles and then reduce them (with gaseous molecular H2 at 673 K) to zero valence Fe without loss of their nano-scale morphology. FIG. 4 shows a high resolution scanning electron micrograph showing that the particle size is homogeneous and close to 7 nm, the diameter of the ferritin shell in which the ferrihydrite precursor was synthesized. The top panel of FIG. 5 exhibits Fe 2p X-ray photoelectron spectroscopy (XPS) data that shows proof of the production of zero valence Fe after reduction. The bottom panel of FIG. 7 exhibits complimentary XPS data that shows that the reduction removes the majority of the N and C, indicating that the bulk of the ferritin is removed from the particle. We do believe, however, that either ferritin or a decomposition of the protein is still present, surrounding some of the particle surface and this acts to prevent aggregation of the small metallic particles. Hence, we will extend this specific development to synthesize zero-valence metal nano-particles with controllable (and homogeneous) dimensions and composition to include Co, Fe, CoxFey and CoxFeyNiz from the respective metal oxide nanoparticles. By employing these alloy combinations we can develop a very wide range in the essential magnetic characteristics of magnetic moment (Fe-Ni alloy system varies from 2.2 μB to 0.2 μB) and magnetic anisotropy (large variation in anisotropy for Co—Fe alloy systems) for the individual clusters. Our synthetic approach addresses these issues, and is rather straightforward, possibly having advantages over more complicated metallic nanoparticle production techniques, such as beam lithography or mass-selected ion beam deposition, which requires highly specialized equipment.

Recent advancements in the use of synchrotron-based magnetic characterization techniques have opened a new opportunity for investigating magnetic interfaces, clusters, and thin films[24]. Soft X-ray magnetic circular dichroism (XMCD)[24-31], which is simply the absorption of circular polarized photons at magnetically interesting transitions, is an element specific probe of magnetic order and structure. It is complementary to the many, more familiar, spin-resolved electron techniques, but instead of resolving or selecting the electron spin, XMCD uses the circular polarized photon selection-rules to probe the wave-function character (spin+symmetry) of the unfilled states. Sensitivity to both electron spin and electron symmetry will be useful for separating the role of these two in spin conductance systems. To obtain the circularly polarized soft X-rays, the MSU/NRL Magnetic Materials X-ray Characterization Facility located at a Beamline at the National Synchrotron Light Source (NSLS) has been modified to simultaneously produce two high intensity soft X-ray beams of opposite circular polarization[32]. Over the past decade, researchers have demonstrated that XMCD can be used in an element specific manner to identify the presence of ferromagnetism[33-37, 6], determine the direction of the magnetic moment of each element [25-27, 36, 37], locate transition temperatures[38], and determine values for the individual spin and orbital contributions to the elemental magnetic moments[29, 38, 39]. The unique element-specific information available from XMCD makes this a powerful tool for understanding nanocluster systems and synthesis. The importance of this type of characterization for the nanoparticle arrays described in this proposal, can be seen from related work done by the P. I. on the valence variation of thin films of Fe3O4 due to overlayer deposition. One of the major interests in Fe3O4 is its use as a half-metallic ferromagnet (HMF) in spin-conductance device multilayer device structures. (Similar applications are apparent in self-assembled ordered arrays.) To maintain these high electron spin polarization values in subsequent layers requires the use of interlayer materials that do not alter the magnetic properties of the Fe3O4. Two candidate materials are TiN and SrTiO (STO). From FIG. 6, we see that a chemical reaction at the TiN/Fe3O4 interface has altered the Fe3O4 near the interfacial region to form FeO, whereas the STO/Fe3O4 interface remains unchanged. The ability to monitor the Fe valence of deeply buried materials is an essential component of this proposal both to confirm our synthesis capabilities and to direct our material processing. The composition of alloy particulates is resolved into their component contributions. Magnetic anisotropy values from orbital and spin moment determinations will assist in our understanding of anisotropy control through particulate alloy generation.

2-D Arrays

The Formation of 2-D Arrays of Protein Cage Assemblies.

We have shown that the protein cages mentioned above can be fabricated into well-ordered 2-D arrays of hexagonally close packed particles, as shown below for both CCMV and ferritin (FIG. 7). This fabrication was achieved through either adsorption of the protein onto freshly cleaved mica, carbon coating and subsequent transfer to a TEM grid or by protein aggregation at a surfactant monolayer at the air-water interface and subsequent transfer to a TEM grid. The close packed lattice of the protein cages imposes a similar geometric ordering on any materials encapsulated within the cage. It has been shown that similar ordering of ferritin proteins and subsequent heat treatment to remove the protein shell does not disrupt the 2-D array of the inorganic nanoparticles [40]. It is our intention to manipulate protein-protein interactions to arrange the protein cages into more complex 2-D arrangements. In addition to the 2-D arrays of protein cage assemblies described above, we propose to incorporate small, functionalized organic particles (dendrimers, 3-6 nm diam.) into the arrays to modulate the communication between the metal particles.

Dendrimer as Interstitial Components of 2-D Arrays

Dendrimers are utilized in two ways. Firstly, they can be easily attached to the surface of the protein cages through chemical modification to direct and spatially define the 2-D arrays. Secondly, spin labeled dendrimers will be used as mediators between magnetic nanoparticles in a 2-D array. Dendrimers are macromolecular compounds that consist of a series of branches around an inner core [41]. The synthesis and structure of poly(amidoamine) (PAMAM) dendrimers, which are commercially available [42], is shown in Scheme 1. PAMAMs have been used in a wide variety of applications ranging from the construction of multilayered films to the binding of DNA. For this project, first (22 nm diam., 8 end groups) through fourth (45 nm diam., 64 end groups) generation dendrimers will be used.

We have developed methodology to surface functionalize PAMAM dendrimers via a thiourea linkage to a variety of surface residues including saccharides, phenols, and TEMPO (2,2,6,6,-tetramethylpiperidine N-oxide free radical) [43]. We have characterized saccharide functionalized PAMAM dendrimers with MW 110,000 g/mol using MALDI-TOF MS and 1H NMR spectroscopy (500 and 600 MHz)[44]. We have characterized TEMPO-labeled dendrimers using MALDI-TOF MS and EPR spectroscopy [44].

By adding two isothiocyanates to the PAMAM dendrimer, we have demonstrated that heterogeneous dendrimer surface functionalization can be achieved. EPR experiments indicate that the distribution of two groups on the dendrimer surface is random (not clustered or maximally separated). A random distribution of surface groups is observed for simultaneous addition of two isothiocyanates and for sequential addition of the two isothiocyanates [45]. Since the diameters of the PAMAM dendrimers are known, the distance between two groups on the dendrimer surface can be calculated (we calculate these values at the 80% probability level). Thus, we have already demonstrated that we can place paramagnetic groups that will moderate properties of the metal particles at known distances on the dendrimer surface in a rapid and reliable way. The synthesis is easily adaptable to accommodate a variety of functional groups onto the dendrimer surface.

Because the dendrimers are significantly smaller than the viral and ferritin cages compounds described above, they can be inserted into the spaces between the cage compounds in the 2-D arrays shown above. In some cases, unfunctionalized dendrimers will be inserted to change the spacing between the cage compounds. In other cases, dendrimers functionalized with free radicals will serve as the interstitial material between the cage compounds and will allow for moderation of the properties of the 2-D arrays. These experiments are described in Specific Aim 4.

Direct characterization of both the nano-particle size distribution and inter-particle spacing and ordering for these 2D arrays can be performed using another X-ray based technique, X-ray resonant magnetic scattering (XRMS). XRMS is the angle dependent specular and off-specular (or diffuse) scattering of circular polarized soft X-rays whose energy is tuned to the absorption edge of a magnetic element present in the material. It combines the element selectivity of X-ray resonant scattering with the magnetic contrast of magnetic circular dichroism, and has been successfully used to separately parameterize the magnetic and chemical roughness of interfaces[30, 46, 47] and can be used to determine inter-particle spacings. Utilizing XRMS will allow us to unfold the complicated topological spin structures present within our magnetic nanoclusters and to identify intercluster interactions for cluster agglomerations and self-assembled structures.

Researchers typically use hard X-ray scattering to obtain information at the atomic scale (atomic positions, interatomic spacing, etc.). The natural length scale for soft X-ray scattering is tens of angstroms, making it ideal for determination of cluster size, inter-cluster distances, and cluster macrostructure. Application of XRMS to our synthesized clusters and cluster arrays will give separate quantitative characterization of the chemical and magnet cluster size distribution, cluster-cluster distances, and cluster-cluster magnetic interactions. These are essential characterization to utilize these systems in magnetic media (FIG. 8).

As an example in FIG. 8 we show the specularly scattered intensity from a single 75 Å EuO film. We observe over 9 integral order Bragg reflections over a 5 order of magnitude variation in scattering intensity. From these types of specular studies and related off-specular (diffuse) studies, a quantitative element and magnetic orientation differentiated determination of inter particle spacings and magnetic order is achievable. This will be an asset as we change both the particulate alloy types and the spin-mediating material.

The 16.5 kDa heat shock protein from Methanococcus janneschii can be cloned into a heterologous expression system. This protein assembles into a 24 subunit protein cage structure both as the native protein when isolated from its native organism and when isolated as a recombinant protein from a heterologous expression system. The interior and exterior surfaces of this protein can be modified to impart unique functionality. The exterior and interior surfaces can be modified through the attachment of organic molecules (fluorphores, metal binding ligands, and drug analogs), peptides, and synthetic polymers to endogenous functional groups. In addition, through genetic engineering of the protein additional functional groups (for example thiols, carboxylic acids and amines) can be added to the interior and exterior of the HSP protein cage for the selective modification of the cage. The engineered thiol groups on the interior can additionally be chemically modified with iodoacetic acid, giving rise to carboxyl groups which are active for the spatially selective oxidation and mineralization of Fe oxides and oxyhydroxides.

The formation of 2-D arrays We have previously shown that ordered 2-D arrays (close packed protein cages) can be made using empty coat protein cages as well as with protein cages which are encapsulating some guest molecule (inorganic nanoparticle, polymer, drug). This can be achieved using both the viral protein cages, the ferritin protein cages, the ferritin-like protein cages and the small heat shock protein cages. The arrays can be formed by spin coating a solution of the protein cages, assembly at the air-water interface beneath Langmuir monolayers, surface attachment of exposed thiol groups to form protein self assembled monolayers and self assembly under concentration above the critical concentration. In this way, we envision using the properties of the cages described above (selective encapsulation/release, gated response to external stimuli, modification of exterior and interior surfaces) to make devices which uniquely exploit the characteristics of protein encapsulated nanomaterials.

Gating: Individual icosahedral assemblies of CCMV undergo a reversible structural transition in response to changes in pH and metal ion concentrations where the virus swells as the pH changes past a threshold value. In the image reconstruction shown below the two conformations (swollen and unswollen) of the gated structure are shown. Particle swelling is a result of expansion at the 60 quasi three-fold axes and causes the opening of holes (approximately 2 nm diameter) in the protein cage. When the pH is raised above a critical value of 6.5, electrostatic repulsion of ionized groups causes the protein expansion. Thus, the swelling can be controlled by changing the solution pH relative to this threshold. Additionally new chemical switches for this gating phenomena that are controlled by an altered pH dependence or by changes in redox state can be engineered into the CCMV structure. We have accomplished this by genetically engineering the coat protein to incorporate disulfide bond formation across the 3-fold interface and by alteration of ionizable groups (acidic to basic) to alter the pH sense of the inherent switch. In the case of the indroduction of thiols we have demonstrated a redox depedent control. Under oxidized conditions, the cage is locked in its closed conformation, whereas tunder reduced conditions the cage can undergo its pH, and metal dependent gating. Likewise our alteration of the ionizable groups at the 3-fold axis has altered the pH dependence of the gating. Control over this gating mechanism will facilitate the uptake and release of material entrapped within the viral protein cage.

Similarly the channels formed at subunit interfaces in ferritin, ferritin-like proteins, and small heat shock protein can be altered and modified by design to control gating. This will therefore control molecular access to and release of materials from the interior of the protein cages.

### BIBLIOGRAPHY

1. Special Issue on “Electronic Cooperation” Science Apr. 21, 2000.

2. G. D. Stucky and J. E. Mac Dougall, “Quantum confinement and host/guest chemistry: Probing a new dimension” Science 247 (1990) 669-678.

3. C. B: Murray, D. J. Norris, and M. G. Bawendi, “Synthesis and characterization on nearly monodisperse CdE (E=S, Se, Te) semiconductor nanocrystallites” J. Am. Chem. Soc. 115 (1993) 8706-8715.

4. E. P. Giannelis, “Organoceramic nanocomposites” in Biomimetic Materials Chemistry, S. Mann, Ed.; (VCH: New York, 1996) p.337-359.

5. S. I. Stupp and P. V. Braun, “Molecular manipulation of microstructures: Biomaterials, ceramics, and semiconductors” Science 277 (1997) 1242-1248.

6. J. W. M. Bulte and R. A. Brooks, “Magnetic nanoparticles as contrast agents for MR imaging” in Scientific and Clinical Applications of Magnetic Carriers, U. Häfeli, W. Schut, J. Teller, and M. Zborowski, Eds.; (Plenum Press: New York, 1997) p.527-543.

7. J. F. Hainfield, “Uranium-loaded apoferritin with antibody attached: Molecular design for uranium neutron-capture therapy” Proc. Natl. Acad. Sci. USA 89 (1992) 11064-11068.

8. L. Brus and D. Eigler, “Devices and desires” Nature 369 (1994) 273-274.

9. S. Mann, Ed.; Biomimetic Materials Chemistry (VCH Publishers: New York, 1996).

10. T. Douglas and S. Mann, “Biomolecules in the synthesis of inorganic solids” in Molecular Biology and Biotechnology, R. A. Meyers, Ed.; (VCH publishers: New York, 1995) p.466-469.

11. T. Douglas, “Biomimetic synthesis of nanoscale particles in organized protein cages” in Biomimetic Approaches in Materials Science, S. Mann, Ed.; (VCH Publishers: New York, 1996) p.91-115.

12. T. Douglas and M. J. Young, “Virus particles as constrained reaction vessels for nanophase mineralization” Nature (1998), 393, 152-154.

13. L. C. Lane, “The bromoviruses” in Handbook of Plant Virus Infection and Comparative Diagnosis, E. Kurstak, Ed.; (Elsevier/North-Holland: Amsterdam, 1981).

14. R. Dasgupta and P. Kaesberg, “Complete nucleotide sequences of the coat protein messenger RNAs of brome mosaic virus and cowpea chlorotic mottle virus” Nucleic Acid Res. 5 (1982) 987-998.

15. P. Ahlquist, “Bromovirus RNA replication and transcription” Curr. Opin. Gen. and Dev. 2 (1992) 71-76.

16. J. M. Fox, J. E. Johnson, and M. J. Young, “RNA/protein interactions in icosahedral virus assembly” Sem. in Vir. 5 (1994).

17. J. M. Fox, X. Zhao, J. A. Speir, and M. J. Young, “Analysis of a salt stable mutant of cowpea chlorotic mottle virus” Virology 222 (1996) 115-122.

18. J. Fox, F. G. Albert, J. Speir, and M. J. Young, “Characterization of a disassembly deficient mutant of cowpea chlorotic mottle virus” Virology 227 (1997) 229-233.

19. Albert, F., J. Fox, and M. J. Young. 1997. Virion swelling is not required for cotranslational disassembly of cowpea chlorotic mottle virus. J. Virol. 71:4296-4299.

20. X. Zhao, J. M. Fox, N. H. Olson, T. S. Baker, and M. J. Young, “In vitro assembly of cowpea chlorotic mottle virus from coat protein expressed in Escherichia coli and in vitro-transcribed viral cDNA” Virology 207 (1995) 486-494.

21. (a) T. Douglas and M. J. Young, “Virus particles as constrained reaction vessels for nanophase mineralization” Nature (1998), 393, 152-154. (b) T. Douglas, E. Strable, D. Willits, A. Aitouchen, M. Libera, M. Young “Protein Engineering of a Viral Cage for Constrained Nano-Materials Synthesis” Advanced Materials(2001), in press.

22. (a) Bozzi, M., Mignogna, G., Stefanini, S., Barra, D., Longhi, C., Valenti, P., and Chiancone, E. (1997). A Novel Non-heme Iron-binding Ferritin Related to the DNA-binding Proteins of the Dps Family in Listeria innocua. J. Biol. Chem. 272, 3259-3265. (b) Yang, X., Chiancone, E., Stefanini, S., IIari, A., and Chasteen, N. D. (2000). Iron oxidation and hydrolysis reactions of a novel ferritin from Listeria innocua. Biochem. J. 349(3), 783-786.

23. S. Gider, D. D. Awschalom, T. Douglas, S. Mann, M. Chaparala, “Classical and Quantum Magnetic Phenomena in Natural and Artificial Ferritin Proteins”, Science, (1995), 268, 77-80.

24. Prinz, G. A., Physics Today, 1995.48: p. 58.

25. Prinz, G. A., Science, 1998. 282: p. 1660.

26. Baibich, M. N., J. M. Broto, A. Fert, F. N. V. Dau, et al., Phys. Rev. Lett., 1988. 61: p. 2472

27. Julliere, M., Phys. Lett. A, 1975. 54: p. 225.

28. Moodera, J., L. Kinder, T. Wong, and R. Meservey, Phys. Rev. Lett., 1995. 74: p. 3273.

29. Prinz, G. A., Science, 1990. 250: p. 1092.

30. Kikkawa, J. M., I. P. Smorchkova, N. Samarth, and D. D. Awschalom, Science, 1997. 277: p. 1284.

31. Kikkawa, J. M. and D. D. Awschalom, Phys. Rev. Lett., 1998.80: p. 4313.

32. Idzerda, Y. U., V. Chakarian, and J. W. Freeland, Sync. Rad. News, 1997

33. Prinz, G. A., Science, 1998.282: p. 1660

34. Baibich, M. N., J. M. Broto, A. Fert, F. N. V. Dau, et al., Phys. Rev. Lett., 1988. 61: p. 2472.

35. Julliere, M., Phys. Lett. A, 1975. 54: p. 225.

36. Kao, C.-C., E. D. Johnson, S. L. Hulbert, J.-M. Brot, et al., Phys. Rev. B (RC), 1994. 50: p. 9599.

37. Chakarian, V., Y. U. Idzerda, G. Meigs, C. T. Chen, et al., eds. Studies of Magnetic Materials with Circular Polarized Soft X-Rays. Applications of Synchrotron Radiation in Industrial, Chemical, and Materials Science, ed. L. J. Terminello, K. L. D. Amico, and D. K. Shuh. 1995, Plenum: N.Y.

38. Chakarian, V., Y. U. Idzerda, K. M. Kemner, J.-H. Park, et al., Phys. Rev. Lett.,: 1999, 82, 1562.

39. Chakarian, V., Y. U. Idzerda, C.-C. Kao, and C. T. Chen, J. Magn. Magn. Mater., 1997. 165:

40. Yamashita, I. (2001). Fabrication of a two-dimensional array of nano-particles using ferritin molecule. Thin Solid Films 393(1-2), 12-18.

41. (a) Matthews, 0. A.; Shipway, A. N.; Stoddart, J. F. “Dendrimers-Branching Out From Curiosities into New Technologies” Prog. Polym. Sci. 1998, 23, 1-56. (b) Zeng, F.; Zimmerman, S. C. “Dendrimers in Supramolecular Chemistry: From Molecular Recognition to Self-Assembly” Chem. Rev. 1997, 97, 1681-1712. (c) Bosman, A. W.; Janssen, H. M.; Meijer, E. W. About Dendrimers: Structure, Physical Properties, and Applications. Chem Rev. 1999, 99, 1665-1688. (d) Hearshaw, M. A.; Moss, J. R. Organometallic and related metal-containing dendrimers. J. Chem. Soc., Chem. Commun. 1999, 1-8. (e) Inoue, K. Prog Polym. Sci. 2000, 25, 453-571.

42. (a) G(0)-through G(4)-PAMAMs are available from Aldrich Chemical Co. in Milwaukee, Wis. (b) Higher generation dendrimers are available from Dendritech, Inc. in Midland, Mich.

43. (a) Woller, E. K.; Cloninger, M. J. “The Lectin-binding Properties of Six Generations of Mannose-functionalized Dendrimers” Org. Lett. 3, (web release date Dec. 11, 2001). (b) Woller, E. K.; Cloninger, M. J. “Mannose Functionalization of a Sixth Generation Dendrimer” Biomacromolecules 2001, 2, 1052.1054.

44. Walter, E. D.; Sebby, K. B.; Singel, D. J.; Cloninger, M. C. “Heterogeneous Dendrimer Surface Functionalization” (manuscript submitted, J. Am. Chem. Soc.).

45. Yamashita, I. (2001). Fabrication of a two-dimensional array of nano-particles using ferritin molecule. Thin Solid Films 393(1-2), 12-18.

46. Chen, C. T., Y. U. Idzerda, H.-J. Lin, N. V. Smith, et al., Phys. Rev. Lett., 1995. 75: p. 152.

47. Freeland, J. W., K. Bussmann, Y. U. Idzerda, and C.-C. Kao, J. Appl. Phys., 1998.83: p. 6290.

48. Gary J. Gerfen, Philip J. Hanna, N. Dennis Chasteen, and David J. Singel. Characterization of the Ligand Environment of Vanadyl Complexes of Apoferritin by Multifrequency Electron Spin Echo Envelope Modulation. Journ. Am. Chem. Soc., 113, (1991) 9513-9519.

49. F. Fominaya, J. Villain, P. Gandit, J. Chaussy, and A. Caneschi, Heat Capacity Anomalies Induced by Magnetization Quantum Tunneling in a Mn12O12-Acetate Single Crystal, Phys. Rev. Lett. 79,1126 (1997).

50. (a) Douglas, T., and Stark, V. T. (2000). Nanophase Cobalt Oxyhydroxide Mineral Synthesized within the Protein Cage of Ferritin. Inorg. Chem. 39,1828-1830. (b) Douglas, T. Bulte, J. W. M., Pankhurst, Q. A., Dickson, D. P. E., Moskowitz, B. M., Frankel, R. B., and Mann, S. (1995). Inorganic-protein interactions in the synthesis of a ferromagnetic nanocomposite. In Hybrid Organic-Inorganic Composites. (J. E. Mark, and P. Bianconi, Eds.), pp. 19-28. American Chemical Society, Washington, D.C. (c) Meldrum, F. C., Douglas, T., Levi, S., Arosio, P., and Mann, S. (1995). Reconstitution of manganese oxide cores in horse spleen and recombinant ferritins. J. Inorg. Biochem. 58, 59-68. (d) Meldrum, F. C., Heywood, B. R., and Mann, S. (1992). Magnetoferritin: In vitro synthesis of a novel magnetic protein. Science 257, 522-523. (e) Meldrum, F. C., Wade, V. J., Nimmo, D. L., Heywood, B. R., and Mann, S. (1991). Synthesis of inorganic nanophase materials in supramolecular protein cages. Nature 349, 684-687.

51. E. Sackman, Science, 1996, 271, 43.

52. S. B. Hwang, J. I. Korebrot, W. Stoeckenius, J. Membr. Biol., 1977, 36, 115-118.

53. N. Ishii, H. Taguchi, M. Yoshida, H. Yoshimura, K. Nagayama, J. Biochem., 1991, 110, 905-908.

54. Ozinskas, A. J.; Bobst, A. M. “Formation of N-Hydroxy-amines of Spin Labeled Nucleosides for 1H NMR Analysis” 63, 1980, 1407.1411.

55. (a) Holmes, C. P.; Jones, D. G. “Reagents for Comdinatorial Organic Synthesis: Development of a New o-Nitrobenzyl Photolabile Linker for Solid Phase Synthesis” J. Org. Chem. 1995, 60, 2318.2319. (b) Rodebaugh, R.; Fraser-Reid, B.; Geysen, H. M. “A New o-Nitrobenzyl Photocleavable Linker for Solid Phase Synthesis” Tetrahedron Lett. 1997, 38, 7653.7656. (c) Smet, M.; Liao, L.-X.; Dehaen, W.; McGrath, D. V. “Photolabile Dendrimers Using o-Nitrobenzyl Ether Linkages” Org. Lett. 2000, 2, 511.513. (d) Dorman, G.; Prestwich, G. D. “Using photolabile ligands in drug discovery and development” Trends in Biotechnology 2000, 18, 64.77.

56. A. T. Yordanov et. al., “Spin Labeled dendrimers in EPR Imaging with Low Molecular Weight Nitroxides” Angew. Chem. Int. ed. Engl. 2001, 40, 2690-2692.

We claim:

1. A composition comprising a 12 subunit protein cage loaded with a first guest material.

2. A composition according to claim 1 wherein said protein cage is a Listeria ferritin.

3. A composition according to claim 1 wherein said first material is a metal.

4. A composition according to claim 3 wherein said metal is iron.

5. A composition according to claim 3 wherein said metal is a mixture of iron and cobalt.

6. A composition according to claim 1 further comprising a plurality of dendrimers associated with said cage.

7. A composition according to claim 6 wherein said dendrimers contain a dopant.

8. A composition comprising a solid support comprising:
a. A plurality of first nanoparticles of a first size, wherein said first nanoparticles comprise a protein cage loaded with a first material;
b. A plurality of second nanoparticles of a second size loaded with a second material.

9. A composition according to claim 8 wherein said first and second materials are the same.

10. A composition according to claim 8 wherein said first and second materials are iron.

11. A composition according to claim 8 wherein said first and second materials are a mixture of iron and cobalt.

12. A composition according to claim 8 wherein said first and second materials are different.

13. A method of manufacturing a composition comprising:
a. Providing first nanoparticles each comprising a 12 subunit protein cage loaded with a first material;
b. Arranging said nanoparticles on a solid support; and
c. Removing said protein cages.

14. A method according to claim 13 further comprising arranging second nanoparticles comprising a protein cage loaded with a second material on said solid support.
